(19) United States
(12) Patent Application Publication
(10) Pub. No.: US 2024/0404251 A1
(43) Pub. Date: Dec. 5, 2024

(54) IMAGE PROCESSING APPARATUS, OPERATION METHOD THEREFOR, INFERENCE APPARATUS, AND LEARNING APPARATUS

(71) Applicant: FUJIFILM Corporation, Tokyo (JP)

(72) Inventor: Shumpei KAMON, Kanagawa (JP)

(73) Assignee: FUJIFILM Corporation, Tokyo (JP)

(21) Appl. No.: 18/805,537

(22) Filed: Aug. 15, 2024

Related U.S. Application Data
(63) Continuation of application No. PCT/JP2022/045861, filed on Dec. 13, 2022.

(30) Foreign Application Priority Data
Feb. 18, 2022 (JP) 2022-024090

(51) Int. Cl. G06V 10/77; G06T 3/4046; G06T 11/00; G06V 10/776; G06V 10/778; G06V 10/82; G06V 20/70

(52) U.S. Cl. CPC G06V 10/7715; G06T 3/4046; G06T 11/00; G06V 10/776; G06V 10/7792; G06V 10/82; G06V 20/70; G06T 2210/41; G06V 2201/03

(57) ABSTRACT

A learning input image is input to a first sub-model to extract a first feature map, and a first output image is output based on the first feature map. The first feature map is input to a second sub-model to extract a second feature map, and a second output image having a higher resolution than the first output image is output. In response to an inference input image being input to a trained learned model, the first output image as an inference result image is output.

### CROSS-REFERENCE TO RELATED APPLICATIONS

This application is a Continuation of PCT International Application No. PCT/JP2022/045861 filed on 13 Dec. 2022, which claims priority under 35 U.S.C § 119 (a) to Japanese Patent Application No. 2022-024090 filed on 18 Feb. 2022. The above application is hereby expressly incorporated by reference, in its entirety, into the present application.

### BACKGROUND OF THE INVENTION

### 1. Field of the Invention

The present invention relates to an image processing apparatus that makes an inference on an image by using machine learning, an operation method for the image processing apparatus, an inference apparatus, and a learning apparatus.

### 2. Description of the Related Art

JP2020-204863A describes “a learning apparatus that gives learning data for learning to a machine learning model having a plurality of layers for analyzing an input image, the machine learning model being for performing semantic segmentation for determining a plurality of classes included in the input image on a pixel-by-pixel basis by extracting, for each layer, features in different ranges of spatial frequencies included in the input image, the learning apparatus including: a reception unit that receives designation of, among a plurality of frequency ranges, at least one of a necessary range estimated to be necessary for learning or an omissible range estimated to be omissible in learning; and a change unit that changes at least one of the machine learning model or the learning data to a mode in accordance with the designation received by the reception unit”.

In addition, JP2020-204863A describes “a decoder network gradually increases the image size of a minimum image feature map output from an encoder network. Then, the gradually increased image feature map and an image feature map output in each layer of the encoder network are combined together to generate a learning output image having the same image size as the learning input image”. Furthermore, JP2020-204863A describes “a learned model performs semantic segmentation on the input image, determines a class and a contour of an object captured in the input image, and outputs an output image as a determination result”.

### SUMMARY OF THE INVENTION

In JP2020-204863A, in the machine learning model for performing semantic segmentation, the decoder network performs processing for gradually increasing the image size. In learning of a machine learning model that performs such segmentation, if learning is performed in such a manner that a high-resolution image is used as correct answer data and a high-resolution image is output also at the time of inference on an unknown image, the determination accuracy at the time of inference by the learned machine learning model is improved. On the other hand, the learned machine learning model that has performed such learning needs to process high-resolution data, and thus, the calculation amount increases. An increase in the calculation amount causes a decrease in the output speed, which is not preferable in a scene in which quick inference is desired, in particular, in a scene in which substantially real-time inference is desired. Thus, it is considered to suppress the calculation amount by using a low-resolution image as the correct answer data. However, if the resolution of the correct answer data is low, the information amount of data to be used for learning decreases, which leads to a decrease in the accuracy of inference. Thus, a technique for causing a machine learning model to learn so as to make an inference on an unknown image at high speed and with high accuracy is desired.

An object of the present invention is to provide an image processing apparatus that achieves higher accuracy of an output result and higher speed of output when an unknown image is input, an operation method for the image processing apparatus, an inference apparatus, and a learning apparatus.

An image processing apparatus according to an aspect of the present invention includes a processor. The processor is configured to output a first output image based on a first feature map extracted by inputting a learning input image to a first sub-model in a learning model including the first sub-model and a second sub-model; output a second output image having a higher resolution than the first output image, based on a second feature map extracted by inputting the first feature map to the second sub-model; calculate an evaluation result by using the second output image; update the learning model by using the evaluation result to set the learning model as a learned model including a first sub-learned model that is the first sub-model that has performed learning and a second sub-learned model that is the second sub-model that has performed learning; and output the first output image as an inference result image based on the first feature map extracted by inputting an inference input image to the first sub-learned model in the learned model.

Preferably, the processor is configured to calculate the evaluation result by comparing the second output image with a learning correct answer image corresponding to the learning input image, and the learning correct answer image is a correct answer label image in which a correct answer label is attached for each of regions constituting the learning correct answer image.

Preferably, the processor is configured to: calculate a first evaluation result as the evaluation result by comparing the first output image with a first correct answer label image as the correct answer label image having a resolution of the first output image, and calculate a second evaluation result as the evaluation result by comparing the second output image with a second correct answer label image as the correct answer label image having the resolution of the second output image; and update the learning model by using the first evaluation result and the second evaluation result.

Preferably, the first correct answer label image is generated by performing resolution reduction processing on the second correct answer label image.

Preferably, the resolution of the second output image is same as a resolution of the learning input image. Preferably, the resolution of the second output image is lower than a resolution of the learning input image.

Preferably, the first sub-model and the second sub-model are constituted by using a convolutional neural network. Preferably, a resolution of the first output image is lower than a resolution of the learning input image.

Preferably, the processor is configured to: further output an intermediate feature map having a higher resolution than the first feature map by using the first sub-model; and further input the intermediate feature map to the second sub-model.

Preferably, the learning input image and the inference input image are medical images. Preferably, the inference input image is an image acquired in time-series order.

Preferably, the processor is configured to: generate report information based on information of the inference result image; generate a report image based on the report information; and perform control to display the report image.

Preferably, the report image is generated to display the report information so as to be superimposed on the inference input image or an image acquired later than the inference input image in time series.

Preferably, the report image is generated so as to display the inference input image or an image acquired later than the inference input image in time series and the report information at positions different from each other.

Preferably, the report information is position information of a specific shape surrounding a region indicating a feature included in the inference input image.

An operation method for an image processing apparatus according to an aspect of the present invention includes: outputting a first output image based on a first feature map extracted by inputting a learning input image to a first sub-model in a learning model including the first sub-model and a second sub-model; outputting a second output image having a higher resolution than the first output image, based on a second feature map extracted by inputting the first feature map to the second sub-model; calculating an evaluation result by using the second output image; updating the learning model by using the evaluation result to set the learning model as a learned model including a first sub-learned model that is the first sub-model that has performed learning and a second sub-learned model that is the second sub-model that has performed learning; and outputting the first output image as an inference result image based on the first feature map extracted by inputting an inference input image to the first sub-learned model in the learned model.

An inference apparatus according to an aspect of the present invention includes a processor. The processor is configured to output a first output image as an inference result image, based on a first feature map extracted by inputting an inference input image to a first sub-learned model in a learned model including the first sub-learned model and a second sub-learned model. The learned model is generated by setting, in a learning model including a first sub-model and a second sub-model, the first sub-model as the first sub-learned model and the second sub-model as the second sub-learned model. The learning model outputs a first output image based on the first feature map extracted based on a learning input image input to the first sub-model, outputs a second output image having a higher resolution than the first output image, based on a second feature map extracted based on the first feature map input to the second sub-model, and is updated by using an evaluation result calculated using the second output image for learning.

A learning apparatus according to an aspect of the present invention includes a processor. The processor is configured to output a first output image based on a first feature map extracted by inputting a learning input image to a first sub-model in a learning model including the first sub-model and a second sub-model; output a second output image having a higher resolution than the first output image, based on a second feature map extracted by inputting the first feature map to the second sub-model; calculate an evaluation result by using the second output image; and update the learning model by using the evaluation result for learning. The resolution of the second output image is lower than the resolution of the learning input image.

According to the present invention, it is possible to achieve higher accuracy of an output result and higher speed of output when an unknown image is input.

### BRIEF DESCRIPTION OF THE DRAWINGS

FIG. 1 is a schematic diagram of an image processing apparatus;

FIG. 2 is a block diagram illustrating functions of a learning apparatus;

FIG. 3 is a block diagram illustrating functions of a learning model;

FIG. 4 is an explanatory diagram illustrating a function of a first sub-model;

FIG. 5 is an explanatory diagram illustrating a function of a second sub-model;

FIG. 6 is an explanatory diagram illustrating functions of an inference apparatus;

FIG. 7 is an explanatory diagram illustrating an example of a learning correct answer image in which small regions are classified by three types of class labels attached thereto;

FIG. 8 is an explanatory diagram illustrating an example of a learning correct answer image in which small regions are classified by two types of class labels attached thereto;

FIG. 9 is an explanatory diagram illustrating an example of mask data to which class labels are attached;

FIG. 10 is an explanatory diagram illustrating functions of an evaluation unit that calculates a plurality of evaluation results by using a plurality of learning correct answer images having resolutions different from each other;

FIG. 11 is an explanatory diagram illustrating an example of the learning model using Unet;

FIG. 12 is an explanatory diagram illustrating an example of the learning model that performs resolution enhancement processing such that a second output image has a higher resolution than a learning input image;

FIG. 13 is an explanatory diagram illustrating an example of the learning model that performs resolution enhancement processing such that the second output image has a lower resolution than the learning input image;

FIG. 14 is a block diagram illustrating functions of a report control unit;

FIG. 15 is an explanatory diagram illustrating functions of the report control unit in a case where position information of a specific shape is generated as report information;

FIG. 16 is an image diagram illustrating an example of a superimposed image on which position information of a specific shape is superimposed;

FIG. 17 is an image diagram illustrating an example of a report image in which the position information of the specific shape is displayed as a sub-image;

FIG. 18 is an explanatory diagram illustrating functions of the report control unit in a case where position information of a small region is generated as the report information;

FIG. 19 is an image diagram illustrating an example of a superimposed image on which the position information of the small region is superimposed;

FIG. 20 is an image diagram illustrating an example of a report image in which the position information of the small region is displayed as a sub-image; and

FIG. 21 is a flowchart illustrating an operation method for the image processing apparatus.

### DESCRIPTION OF THE PREFERRED EMBODIMENTS

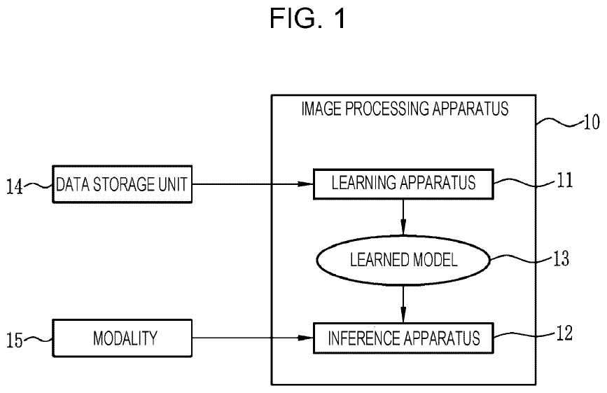

As illustrated in FIG. 1, an image processing apparatus 10 includes a learning apparatus 11 and an inference apparatus 12. The learning apparatus 11 and the inference apparatus 12 are communicably connected to each other in a wired manner or a wireless manner via a network. The network is, for example, the Internet or a local area network (LAN).

By causing a learning model 30 to learn in the learning apparatus 11, the image processing apparatus 10 sets the learning model 30 as a learned model 13 that infers a membership probability with respect to a small region of an image and that extracts a region of interest that is a region to be focused included in the image. The learned model 13 is transmitted to the inference apparatus 12. In response to an unknown image being input to the inference apparatus 12, a region of interest included in the unknown image is extracted. The small region of the image refers to a pixel or a group of pixels constituting the image.

The learning model 30 is a model that performs feature extraction and resolution enhancement processing on an input image. A control unit (not illustrated), which is a processor included in the image processing apparatus 10, inputs a learning input image 21 from a learning data set 20 stored in a data storage unit 14 to the learning model 30. The learning model 30 outputs a first output image 42 in which a feature of the learning input image 21 is extracted and a second output image 52 having a higher resolution than the first output image 42. The learning apparatus 11 updates the learning model 30 to the learned model 13 by using the second output image 52, and transmits the trained learned model 13 to the inference apparatus 12. In response to an inference input image 121, which is an unknown image, being input from a modality 15, the learned model 13 performs, on the inference input image 121, inference processing for performing at least feature extraction on the image to output the first output image 42.

The data storage unit 14 may be provided either outside or inside the image processing apparatus 10. In a case where the data storage unit 14 is provided outside the image processing apparatus 10, the learning data set 20 is input from the data storage unit 14 to the learning apparatus 11 via the network. In a case where the data storage unit 14 is provided inside the image processing apparatus 10, the learning data set 20 is read to the learning apparatus 11 and input to the learning model 30.

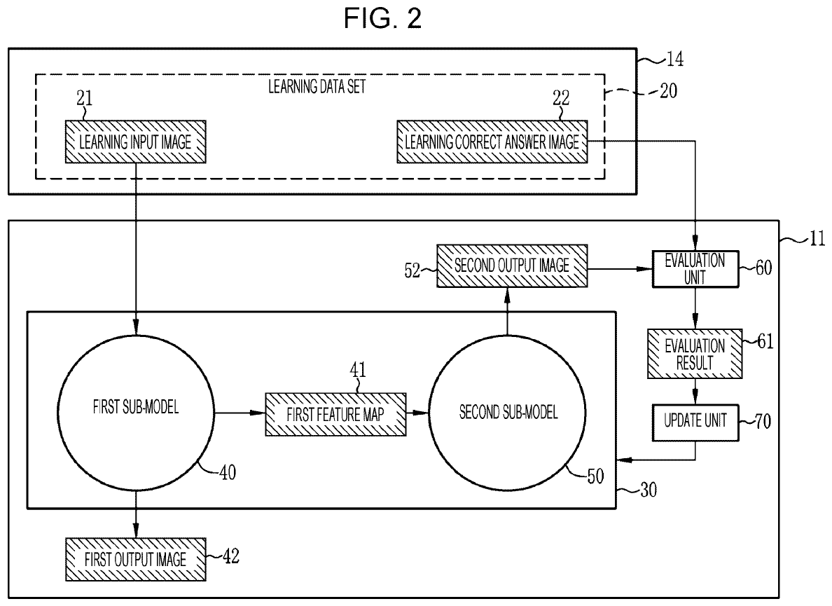

A specific configuration of the learning apparatus 11 will be described. As illustrated in FIG. 2, the learning apparatus 11 includes the learning model 30, an evaluation unit 60, and an update unit 70. In response to the learning input image 21 being input, the learning model 30 outputs the first output image 42 and the second output image 52 by using machine learning. The learning model 30 includes a first sub-model 40 for extracting a feature of the input image and a second sub-model 50 for performing resolution enhancement processing on input image data. The learning input image 21 from the learning data set 20 stored in the data storage unit 14 is input to the first sub-model 40. Note that the number and configuration of sub-models of the learning model 30 are not limited to those described above as long as the entire model performs feature extraction and resolution enhancement processing on an input image.

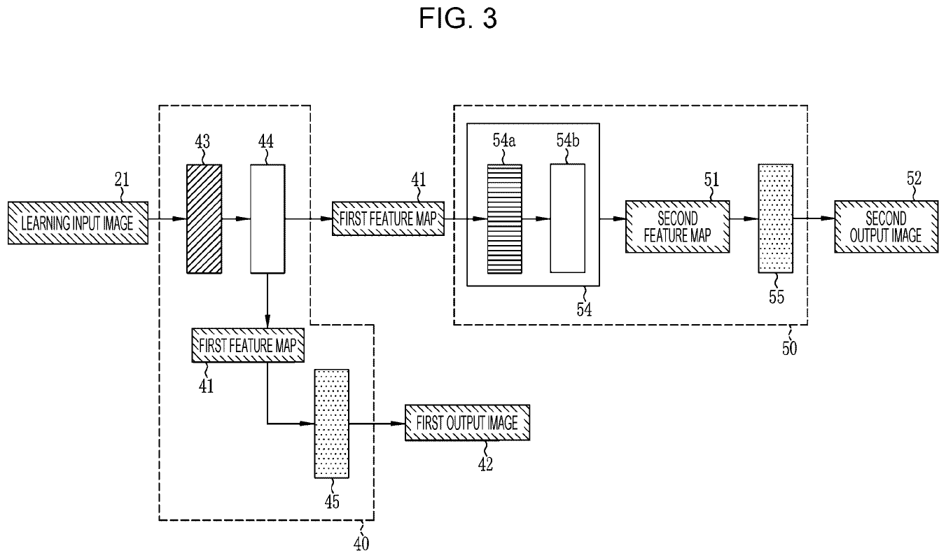

The first sub-model 40 and the second sub-model 50 are preferably configured by using convolutional neural networks having a layered structure as illustrated in FIG. 3. The learning input image 21 is input to an input layer 43 of the first sub-model 40. Subsequently, in a first intermediate layer 44, which is an intermediate layer of the first sub-model 40, a convolutional operation using a plurality of filters is performed at least once to extract a first feature map 41 in which a feature of the learning input image 21 is extracted. The first feature map 41 is input to a first output layer 45 and the second sub-model 50.

The first intermediate layer 44 has one or more convolutional layers. In the convolutional layer, filters are applied to image data that is input, and a feature map indicating positions where patterns of the filters are present is extracted from the input image data. The filter is also referred to as a convolution kernel. Note that the feature map is also included in the image data input to the convolutional layer. The same number of feature maps as the plurality of filters used in one convolutional layer are extracted.

The first intermediate layer 44 may or may not have a pooling layer. The pooling layer is a layer that summarizes values related to a local region of the input image data and performs resolution reduction processing of the image data. The first intermediate layer 44 may be constituted by one convolutional layer, but is preferably constituted by a plurality of convolutional layers and pooling layers from the viewpoint of improving the accuracy and increasing the speed of feature extraction.

The first feature map 41 is a feature map output from the convolutional layer or the pooling layer at the most subsequent stage of the first intermediate layer 44. In a case where the first intermediate layer 44 is constituted by a plurality of convolutional layers and pooling layers, among feature maps extracted in the first intermediate layer 44, a feature map extracted from the layer at the most subsequent stage is the first feature map 41, and a feature map extracted from a layer at a stage before the layer from which the first feature map 41 is extracted is a first intermediate feature map. Modifications of constituting the first intermediate layer 44 by a plurality of layers will be described later.

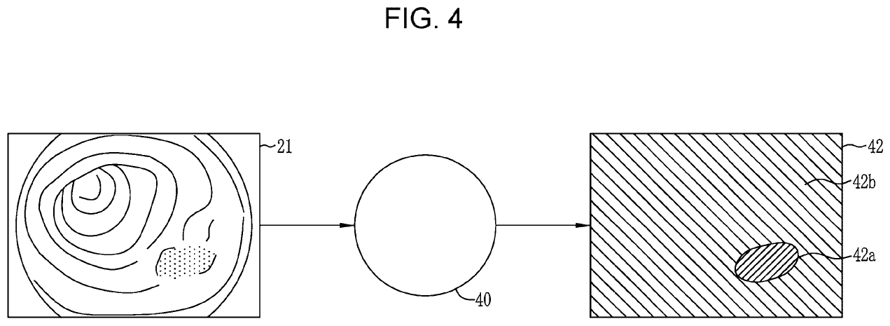

The first feature map 41 extracted from the first intermediate layer 44 is input to the first output layer 45. In the first output layer 45, one first output image 42 is output from a plurality of first feature maps 41 by using an activation function. As illustrated in FIG. 4, in the first output image 42, the membership probability for each region with respect to an input image (the learning input image 21 in FIG. 4) is calculated, and the regions are classified. For example, the regions are classified into a region of interest 42a and a region 42b other than the region of interest.

The first feature map 41 extracted from the first intermediate layer 44 is further transmitted to a second intermediate layer 54 of the second sub-model 50. The second intermediate layer 54 at least performs processing for increasing the resolution of the first feature map 41 and extracts a second feature map 51 (see FIG. 3).

The second intermediate layer 54 has one or more upsampling layers 54a. The upsampling layer 54a performs enlargement processing (resolution enhancement processing) of a feature map. In addition, the second intermediate layer 54 preferably further has a convolutional layer 54b. One upsampling layer 54a and one convolutional layer 54b may be provided, but a plurality of upsampling layers 54a and convolutional layers 54b are preferably provided from the viewpoint of the accuracy of feature extraction.

Examples of a method of the resolution enhancement processing include upsampling in which pixel values of pixels constituting the feature map are arranged at intervals of some pixels and pixel values therebetween are interpolated, and upconvolution in which upsampling without interpolation of pixel values and convolution are combined. The upsampling is also referred to as unpooling, and the upconvolution is also referred to as transposition convolution or deconvolution. Note that the second intermediate layer 54 may be configured without the upsampling layer 54a. In this case, the second intermediate layer 54 performs the resolution enhancement processing by, for example, a shift-and-stitch method.

The second feature map 51 is a feature map output from the convolutional layer at the most subsequent stage of the second intermediate layer 54. In a case where the second intermediate layer 54 is constituted by the plurality of upsampling layers 54a and convolutional layers 54b, among feature maps extracted in the second intermediate layer 54, a feature map extracted from the layer at the most subsequent stage is the second feature map 51, and a feature map extracted from a layer at a stage before the layer from which the second feature map 51 is extracted is a second intermediate feature map. That is, the second feature map 51 is a feature map extracted from the layer at the most subsequent stage among feature maps extracted in the second intermediate layer 54. Modifications of constituting the second intermediate layer 54 by a plurality of layers will be described later.

The second feature map 51 extracted from the second intermediate layer 54 is input to a second output layer 55. In the second output layer 55, one second output image 52 is output from a plurality of second feature maps 51 by using the activation function as in the first output layer 45. Since the resolution enhancement processing of the first feature map 41 is performed by using the second intermediate layer 54, the second output image 52 has a higher resolution than the first output image 42.

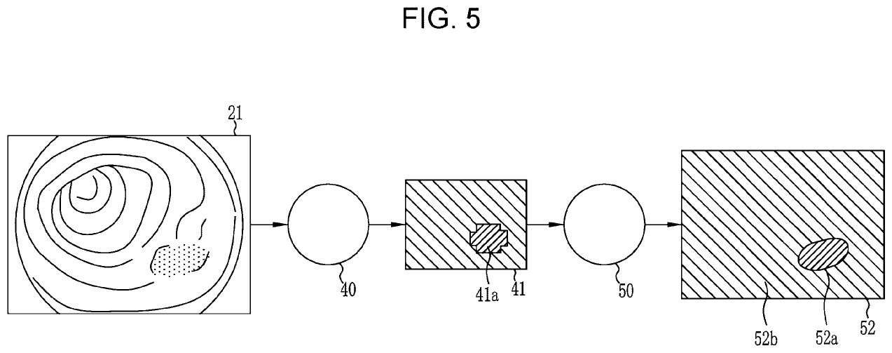

As illustrated in FIG. 5, the second output image 52 indicates a result of performing the resolution enhancement processing on the first feature map 41 in which a feature (a region of interest 41a in FIG. 5) of an input image (the learning input image 21 in FIG. 5) is extracted, and is divided into, for example, a region of interest 52a and a region 52b other than the region of interest. In the specific example in FIG. 5, an example is illustrated in which the first intermediate layer 44 of the first sub-model 40 performs the resolution reduction processing on the learning input image 21, and the second intermediate layer 54 of the second sub-model 50 performs the resolution enhancement processing such that the first feature map 41 has substantially the same resolution as the learning input image 21.

Note that as long as the second output image 52 has a higher resolution than the first output image 42, the second output image 52 may have a lower resolution than the learning input image 21, may have the same resolution as the learning input image 21, or may have a higher resolution than the learning input image 21.

The second output image 52 is transmitted to the evaluation unit 60 (see FIG. 2). The evaluation unit 60 outputs an evaluation result 61 by using the second output image 52. For example, in a case of supervised learning, the evaluation unit 60 evaluates the output accuracy of the entire learning model 30 by outputting a loss that is a degree of a difference between the second output image 52 and a learning correct answer image 22 by using a loss function (also referred to as an error function) that is a model for evaluation. In this case, the evaluation result 61 is a loss (also referred to as an error) calculated by the evaluation unit 60 by using the loss function. As the evaluation result 61 is closer to 0, the difference between the second output image 52 and the learning correct answer image 22 is smaller, and the output accuracy of the learning model 30 is higher.

The learning correct answer image 22 is an image in which the position of a region of interest is indicated in advance, an image in which one type of class label (correct answer label) among a plurality of types of class labels is attached for each small region, or the like. Specific examples of the learning correct answer image 22 will be described later.

The update unit 70 updates the learning model 30 in accordance with the evaluation result calculated by the evaluation unit 60. As a specific example, for example, parameters (weights and biases) of the networks of the first sub-model 40 and the second sub-model 50 are updated such that the loss approaches 0. The update unit 70 updates the parameters of the networks so as to minimize the loss by using, for example, a stochastic gradient descent method. In this case, the learning rate defines the magnitude of the update amount, and as the learning rate is higher, the width of change of the parameters is larger. Note that the update method is not limited to this.

Note that semi-supervised learning may be performed by using a learning image without a correct answer label in addition to the learning correct answer image 22 with the correct answer label. In this case, the evaluation unit 60 sets, as an objective function, a certain condition satisfied by the learning image without a correct answer label in a loss function used for supervised learning, and sets, as an evaluation result, an arithmetic value calculated from a function obtained by adding the loss function and the objective function. The update unit 70 may update the parameters so as to minimize the arithmetic value calculated from the function obtained by adding the loss function and the objective function.

The calculation of the evaluation result 61 by the evaluation unit 60 and the update of the learning model 30 by the update unit 70 are repeatedly continued until the evaluation result 61 reaches a preset value. The preset value may be a value within a certain range, or may be greater than or equal to a certain threshold value or less than the threshold value.

If the evaluation result 61 of the evaluation unit 60 reaches the preset value, the learning model 30 is set as the learned model 13 including a first sub-learned model that is the learned first sub-model 40 and a second sub-learned model that is the learned second sub-model 50. The learned model 13 finally generated by the learning apparatus 11 has the same configuration as the learning model 30. For example, if the learning model 30 has the configuration illustrated in FIG. 3, the learned model 13 has the same configuration.

The learned model 13 is transmitted from the learning apparatus 11 to the inference apparatus 12 (see FIG. 1). The learned model 13 transmitted from the learning apparatus 11 to the inference apparatus 12 includes the first sub-learned model that is the learned first sub-model. The learned model 13 transmitted to the inference apparatus 12 may be constituted by the first sub-learned model and the second sub-learned model, but is preferably constituted by only the first sub-learned model. This is because, from the viewpoint of hardware, there is an advantage that a memory can be saved by omitting the second sub-learned model from the inference apparatus 12.

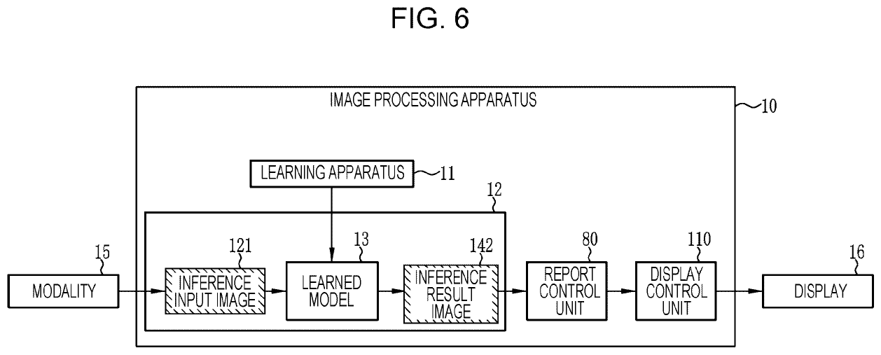

As illustrated in FIG. 6, the inference input image 121 is input from the modality 15 to the inference apparatus 12. The inference input image 121 is input to the input layer 43 of the first sub-learned model in the learned model 13. Subsequently, the first intermediate layer 44 of the first sub-learned model extracts first feature maps 41, and the first output layer 45 outputs one first output image 42 from the plurality of first feature maps 41 (see FIG. 3). In this example, the first output image 42 output from the first sub-learned model is an inference result image 142. That is, in response to the inference input image 121 being input, the learned model 13 outputs the first output image 42 as the inference result image 142.

As in this example, by the learning model 30 performing learning such that the second output image 52 has a higher resolution than the first output image 42, the output accuracy of the learned model 13 is improved. Furthermore, as in this example, by providing the output layer in the first sub-model (the first sub-learned model in the learned model 13), the first output image 42 can be output quickly. That is, with the configuration described in this example, it is possible to promote an increase in the speed of inference processing on an unknown image.

In a machine learning model that performs two different operations, such as feature extraction in one model and resolution enhancement processing in the other model, in general, an output layer is not provided between the one model and the other model. Thus, as in this example, the learned model 13 obtained by learning of the learning model 30 in which the second sub-model that performs the resolution enhancement processing is provided with the output layer and the first sub-model that performs the feature extraction is also provided with the output layer can perform inference processing that is faster than a general machine learning model and achieves high recognition accuracy. That is, the learned model 13 in this example can achieve substantially real-time output with high accuracy in response to input of an unknown image.

In a case where the learned model 13 is constituted by the first sub-learned model and the second sub-learned model, when the inference result image 142 is output, the second output image may be output from the second sub-learned model, but the second output image is not used to generate report information. When the inference input image 121 is input to the learned model 13, it is preferable to use only the first sub-learned model and not to output the second output image without using the second sub-learned model. Although in a case of inputting the inference input image 121, which is an unknown image, to the learned model 13, sufficiently quick output of the first output image 42 can be achieved by installing the first sub-learned model in the inference apparatus 12, by outputting the inference result image 142 by using only the first sub-learned model, the arithmetic processing in the inference apparatus 12 can be performed at higher speed.

In addition, in a case where the second sub-learned model is not used when the inference result image 142 is output, the first feature map extracted by the first sub-learned model is preferably not input to the second sub-learned model.

The evaluation unit 60 preferably compares the second output image 52 with the learning correct answer image 22 and calculates the evaluation result 61 that evaluates the accuracy of the calculation of the membership probability or the classification for each small region. The learning correct answer image 22 used in the learning apparatus 11 is preferably a correct answer label image in which a correct answer label is attached to each region constituting the learning correct answer image 22. The correct answer label refers to a class label indicating “correct answer” attached to each small region constituting the learning correct answer image 22.

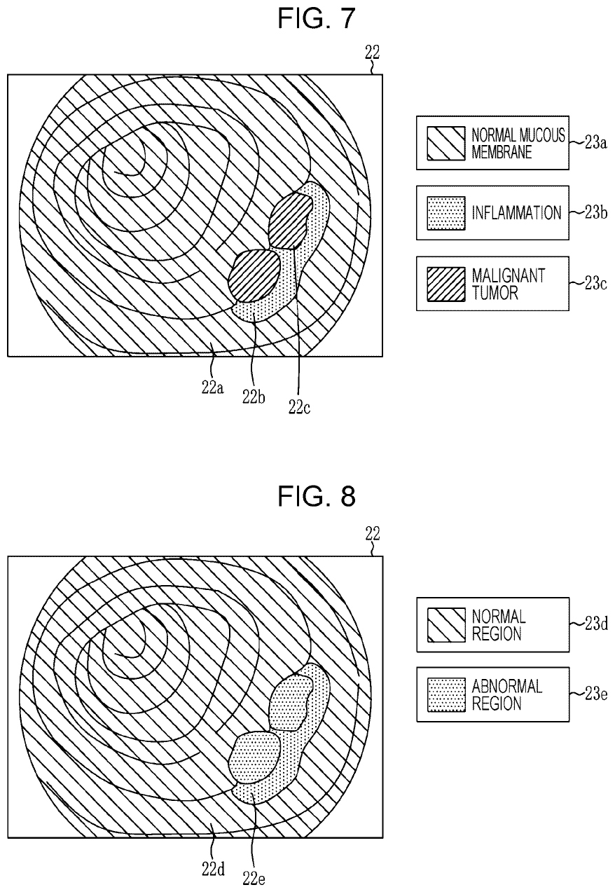

For example, in a specific example in FIG. 7, a correct answer label 23a of “normal mucous membrane”, a correct answer label 23b of “inflammation”, and a correct answer label 23c of “malignant tumor” are respectively attached to a small region 22a, a small region 22b, and a small region 22c constituting the learning correct answer image 22.

In addition, as illustrated in a specific example in FIG. 8, the correct answer labels may be attached by dividing the learning correct answer image 22 into a region of interest and a region other than the region of interest. In the specific example in FIG. 8, a correct answer label 23d of “normal region” as the region other than the region of interest and a correct answer label 23e of “abnormal region” as the region of interest are respectively attached to a small region 22d and a small region 22e constituting the learning correct answer image 22. Examples of the correct answer labels are not limited to these.

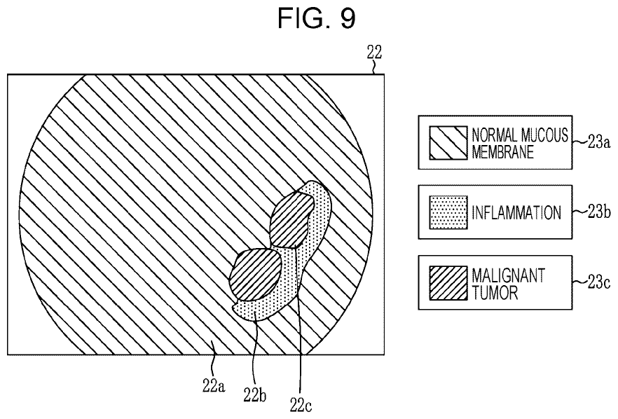

In the specific examples in FIGS. 7 and 8, the learning correct answer image 22 is illustrated in which the correct answer label is attached to the small region corresponding to the learning input image 21 in which the structure of folds or the like of a mucous membrane or redness of inflammation is visually distinguishable. On the other hand, as illustrated in FIG. 9, the learning correct answer image 22 is preferably mask data in which the structure of folds or the like of a mucous membrane, redness of inflammation, or the like is not visually distinguishable and small regions to which correct answer labels are attached are divided from one another by different colors. In the specific example in FIG. 9, the learning correct answer image 22 is illustrated in which the correct answer labels 23a, 23b and 23c are attached to the small regions 22a, 22b, and 22c, respectively, as in FIG. 7, and only the class to which each small region belongs is distinguishable.

In a case of using the learning correct answer image 22 illustrated in the specific examples in FIGS. 7 to 9, the learning model 30 is a model for segmentation, and, in the first output image 42 and the second output image 52, class labels are predicted for the small regions constituting the learning input image 21. With the above configuration, the learned model 13 can be a model for performing segmentation on an unknown image and detecting a region of interest with high accuracy and at high speed.

The region of interest is a region to which a user pays attention. For example, in a case of a medical image, the region of interest refers to a region indicating an abnormality such as a malignant tumor, a benign tumor, a polyp, inflammation, bleeding, vascular irregularity, ductal irregularity, hyperplasia, dysplasia, an injury, or a fracture, a region that is not normal in a living body or a region where treatment is performed on a living body, such as a scar, a surgical scar, or a foreign substance such as a medical fluid, a fluorescent dye, an artificial joint, an artificial bone, or gauze, in the medical image. In addition, in a case of an image in which a product of a machine tool is a subject, for example, the region of interest is a region indicating an abnormality such as a crack, a break, or a scratch of the product. Note that examples of the region of interest are not limited to these.

In addition, the learning correct answer image 22 may be an image in which the correct answer label is attached only to the region of interest. In this case, the learning model 30 may output the class label only for the small region that is the region of interest, without outputting the class label for small regions other than the region of interest.

Note that the classification of the small regions and the assignment of the class labels, which are performed in advance on the learning correct answer image 22, may be performed by a user or may be performed by machine learning installed in an apparatus other than the image processing apparatus 10. The user is, for example, a doctor or the like skilled in medical image diagnosis.

It is preferable that the evaluation result be further calculated by comparing the learning correct answer image 22 with the first output image 42 in addition to comparing the learning correct answer image 22 with the second output image 52. That is, FIG. 2 illustrates a specific example in which the evaluation result 61 is calculated by comparing the learning correct answer image 22 with the second output image 52, but in addition to this, it is preferable that an evaluation result be further calculated by comparing the learning correct answer image 22 with the first output image 42.

In this case, as the learning correct answer image 22, the learning correct answer image 22 having two types of resolutions, which are the learning correct answer image 22 (first correct answer label image) having the resolution of the first output image 42 and the learning correct answer image 22 (second correct answer label image) having the resolution of the second output image 52, is included in the learning data set 20. Note that the resolution of the first correct answer label image is preferably as close to that of the first output image 42 as possible, and more preferably equal to that of the first output image 42. Similarly, the resolution of the second correct answer label image is preferably as close to that of the second output image 52 as possible, and more preferably equal to that of the second output image 52. The resolutions of the first correct answer label image and the second correct answer label image are different from each other, and the resolution of the second correct answer label image is higher than the resolution of the first correct answer label image.

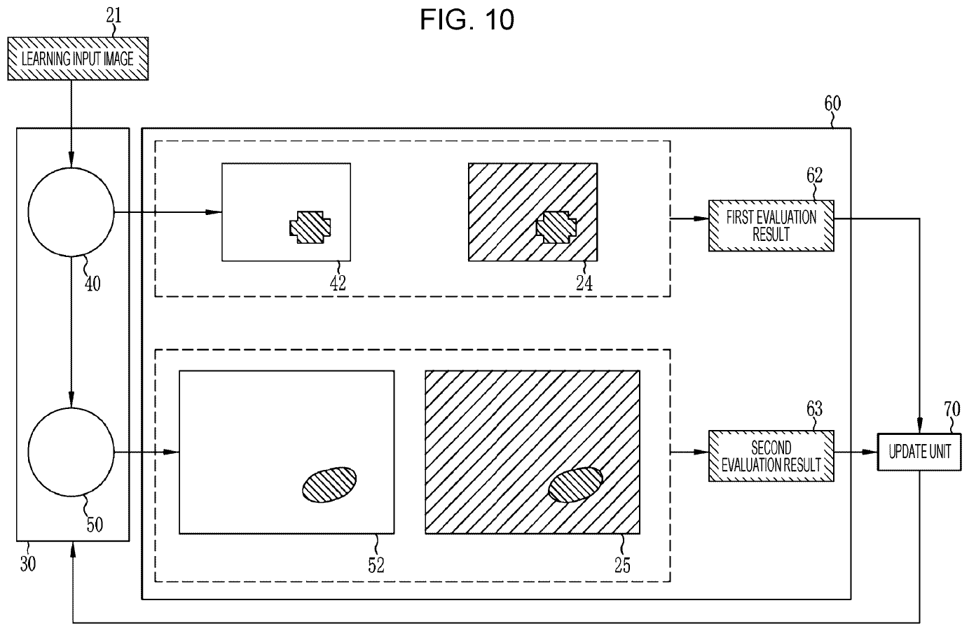

In this example, as illustrated in FIG. 10, the evaluation unit 60 compares the first output image 42 output by the first sub-model 40 in response to the learning input image 21 being input to the first sub-model with a first correct answer label image 24, and calculates a first evaluation result 62 as an evaluation result. Furthermore, the evaluation unit 60 compares the second output image 52 output by the second sub-model 50 with a second correct answer label image 25, and calculates a second evaluation result 63 as an evaluation result.

The calculated first evaluation result 62 and second evaluation result 63 are input to the update unit 70. The update unit 70 updates the learning model 30 based on the first evaluation result 62 and the second evaluation result 63. The first evaluation result 62 is a loss indicating a difference between the first output image 42 and the first correct answer label image 24, and the second evaluation result 63 is a loss indicating a difference between the second output image 52 and the second correct answer label image 25. With the above configuration, the learning model 30 can be updated with two types of evaluation results, and thus, the learning accuracy can be further improved.

Although the first correct answer label image 24 and the second correct answer label image 25 may be generated one by one, the first correct answer label image 24 is preferably generated by performing resolution reduction processing on the second correct answer label image 25. In this case, a first correct answer label image generation unit (not illustrated) may be provided in the image processing apparatus 10, and the first correct answer label image generation unit may generate the first correct answer label image 24 by reducing the resolution of the second correct answer label image 25, or an apparatus other than the image processing apparatus 10 may generate the first correct answer label image 24 by reducing the resolution of the second correct answer label image 25. With the above configuration, it is possible to generate the second correct answer label image 25 at low cost without newly generating the first correct answer label image 24.

If the second output image 52 output from the second sub-model 50 has a higher resolution than the first output image 42 output from the first sub-model 40, the first sub-model 40 may output the first output image 42 by performing an operation for reducing the resolution of the learning input image 21 or may output the first output image 42 having the same resolution as the learning input image 21. In addition, the second sub-model 50 may output the second output image 52 having the same resolution as the learning input image 21, may output the second output image 52 having a higher resolution than the learning input image 21, or may output the second output image 52 having a lower resolution than the learning input image 21.

Combinations of processing performed in the first sub-model 40 and the second sub-model 50 will be described.

(1) The learning model 30 in which the first sub-model 40 performs feature extraction and resolution reduction processing, and the second sub-model 50 performs resolution enhancement processing such that the second output image 52 has the same resolution as the learning input image 21.

(2) The learning model 30 in which the first sub-model 40 performs feature extraction and resolution reduction processing, and the second sub-model 50 performs resolution enhancement processing such that the second output image 52 has a higher resolution than the learning input image 21.

(3) The learning model 30 in which the first sub-model 40 performs feature extraction and resolution reduction processing, and the second sub-model 50 performs resolution enhancement processing such that the second output image 52 has a lower resolution than the learning input image 21 (however, the second output image 52 has a higher resolution than the first output image 42).

(4) The learning model 30 in which the first sub-model 40 does not perform resolution reduction processing, and the second sub-model 50 performs resolution enhancement processing such that the second output image 52 has a higher resolution than the learning input image 21.

The first output image 42 preferably has a lower resolution than the learning input image 21. In a case where the first output image 42 has a lower resolution than the learning input image 21, the output speed of the first output image 42 of the finally generated learned model 13 is higher than in a case where the first output image 42 has the same resolution as the learning input image 21. That is, by the first sub-model 40 performing the resolution reduction processing, the inference processing speed of the trained learned model 13 can be improved. In the examples of the learning models 30 of (1) to (4) described above, in the learning models 30 of (1) to (3) in which the first sub-model performs the resolution reduction processing, the first output image 42 is output faster than in the learning model 30 of (4).

In addition, by the first sub-model 40 performing the resolution reduction processing, it is possible to extract the first feature map 41 in which information in a wider range in the image is aggregated. For example, in a case where convolution processing is performed on a high-resolution image and an edge is extracted from the image, it may be difficult to accurately recognize whether a small region including the extracted edge is a normal mucous membrane or a polyp and to perform classification. Regarding such a problem, as a result of further aggregating information by reducing the resolution of a feature map obtained by convolution and aggregating information in a wide range by repeatedly performing convolution, it may be possible to determine that the edge is a polyp.

By extracting the first feature map 41 in which information in a wide range is aggregated through the resolution reduction processing in the first sub-model 40 and enhancing the resolution of the first feature map 41 in which information is aggregated in the second sub-model 50, it is possible to restore the position information of the once-aggregated information of a local feature in the entire image and to update the learning model 30 in such a manner that the extracted feature and the position information thereof become accurate. The learned model 13 that has performed such learning can recognize an unknown high-resolution image with high accuracy. In particular, in segmentation in which classification is performed for each small region of an image, the recognition accuracy can be improved by learning for making the position information of a feature accurate.

As the resolutions of the second feature map 51 and the second output image 52 based on the second feature map are higher, learning can be performed to improve the output accuracy of the learning model 30. Accordingly, the accuracy of the inference processing of the learned model 13 is improved. In the examples of the learning models 30 of (1) to (4) described above, the learning models 30 of (2) and (4) in which the second sub-model 50 performs the resolution enhancement processing such that the second output image 52 has a higher resolution than the learning input image 21, have higher output accuracy with respect to the learning input image 21 than the learning models 30 of (1) and (3).

On the other hand, in learning of a learning model using segmentation, in general, as the resolution of an image to be finally output is higher, overlearning is likely to occur due to an increase in parameters to be used for learning. Thus, by outputting the second output image 52 having a lower resolution than the learning input image 21, learning can be stabilized, and overlearning can be suppressed. In this manner, if the second output image 52 has a higher resolution than the learning input image 21, there is a trade-off relationship between higher accuracy of inference with respect to the learning input image 21 and overlearning that reduces the recognition accuracy with respect to an unknown image. By providing, in the learning apparatus 11, among the examples of the learning model 30 of (1) to (4) described above, the learning model 30 of (3) in which the second sub-model 50 performs the resolution enhancement processing such that the second output image 52 has a lower resolution than the learning input image 21, it is possible to provide the learning apparatus 11 capable of suppressing overlearning.

In addition, in addition to the first feature map 41 extracted from the first sub-model 40, an intermediate feature map (first intermediate feature map) is preferably input to the second sub-model 50. As the learning model 30 having such a configuration, ResNet (Residual Network) and Unet (U-shaped Network) are known.

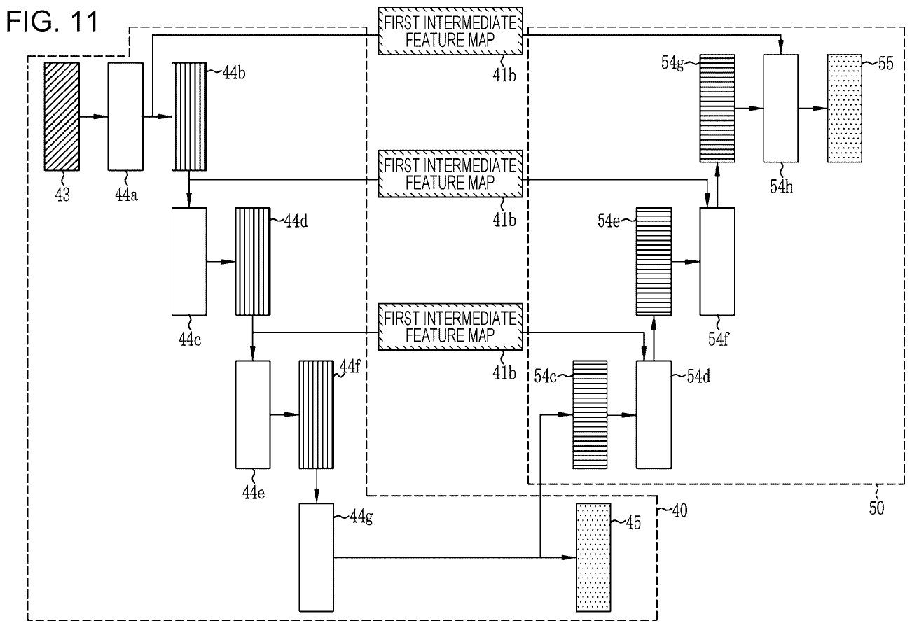

A case where Unet is used for the learning model 30 will be described with reference to a specific example illustrated in FIG. 11. The first intermediate layer 44 (see FIG. 3) of the first sub-model 40 has a plurality of convolutional layers 44a, 44c, 44e, and 44g and a plurality of pooling layers 44b, 44d, and 44f.

The pooling layer 44b performs downsampling of a feature map input from the convolutional layer 44a to reduce the resolution of the feature map. Similarly, the pooling layer 44d reduces the resolution of a feature map input from the convolutional layer 44c, and the pooling layer 44f reduces the resolution of a feature map input from the convolutional layer 44e. The pooling layers 44b, 44d, and 44f provide robustness to position information of an extracted feature and further contribute to extraction of a feature necessary for class classification.

In the first sub-model 40 illustrated in FIG. 11, a feature map extracted from the convolutional layer 44g, which is the layer at the most subsequent stage, is the first feature map 41. Each of the feature maps extracted from the convolutional layer 44a and the pooling layers 44b and 44d is a first intermediate feature map 41b.

The second intermediate layer 54 (see FIG. 3) of the second sub-model 50 has a plurality of upsampling layers 54c, 54e, and 54g and a plurality of convolutional layers 54d, 54f, and 54h. The upsampling layer 54c enhances the resolution of the first feature map 41 input from the convolutional layer 44g of the first sub-model 40. Similarly, the upsampling layer 54e enhances the resolution of a feature map input from the convolutional layer 54d, and the upsampling layer 54g enhances the resolution of a feature map input from the convolutional layer 54f.

In the second sub-model 50 illustrated in FIG. 11, a feature map extracted from the convolutional layer 54h, which is the layer at the most subsequent stage, is the second feature map 51. Each of the feature maps extracted from the convolutional layers 54d and 54f other than the convolutional layer 54h and feature maps extracted from the upsampling layers 54c, 54e, and 54g is a second intermediate feature map.

In Unet, layers for convolution of intermediate feature maps having similar resolutions are paired, and an intermediate feature map (the first intermediate feature map 41b) extracted by a sub-model that performs downsampling is input to a paired layer of a sub-model that performs upsampling. The layers to be paired in the specific example in FIG. 11 are as follows. (1; First Layer) A layer of the convolutional layer 44a and the pooling layer 44b and a layer of the upsampling layer 54g and the convolutional layer 54h. (2; Second Layer) A layer of the convolutional layer 44c and the pooling layer 44d and a layer of the upsampling layer 54e and the convolutional layer 54f. (3; Third Layer) A layer of the convolutional layer 44e and the pooling layer 44f and a layer of the upsampling layer 54c and the convolutional layer 54d. Note that the resolution reduction processing is performed in a stepwise manner from the first layer to the third layer in the first sub-model 40, and the resolution enhancement processing is performed in a stepwise manner from the third layer to the first layer in the second sub-model 50.

As in the specific example illustrated in FIG. 11, in the first layer, the first intermediate feature map 41b extracted by the convolutional layer 44a is input to the convolutional layer 54h. In the second layer, the first intermediate feature map 41b extracted by the pooling layer 44b is input to the convolutional layer 54f. In the third layer, the first intermediate feature map 41b extracted by the pooling layer 44d is input to the convolutional layer 54d.

In this manner, by inputting the first intermediate feature maps 41b extracted by the first sub-model 40 to the second sub-model 50, it is possible to easily recover spatial resolutions that have been lost once in the process of downsampling, which is considered to be difficult, and to perform high-accuracy learning. In addition, the spatial resolutions are recovered by combining the first intermediate feature map 41b and the second intermediate feature map, for example, by addition processing.

Note that the intermediate feature map may be transferred in the paired layers as in Unet, and the resolution of the first intermediate feature map extracted by the first sub-model 40 may be enhanced, and the first intermediate feature map having the enhanced resolution may be input to the second sub-model 50. That is, in Unet, the intermediate feature map may be transferred to a layer other than the paired layer. Also by this method, it is possible to easily recover the spatial resolutions at the time of upsampling.

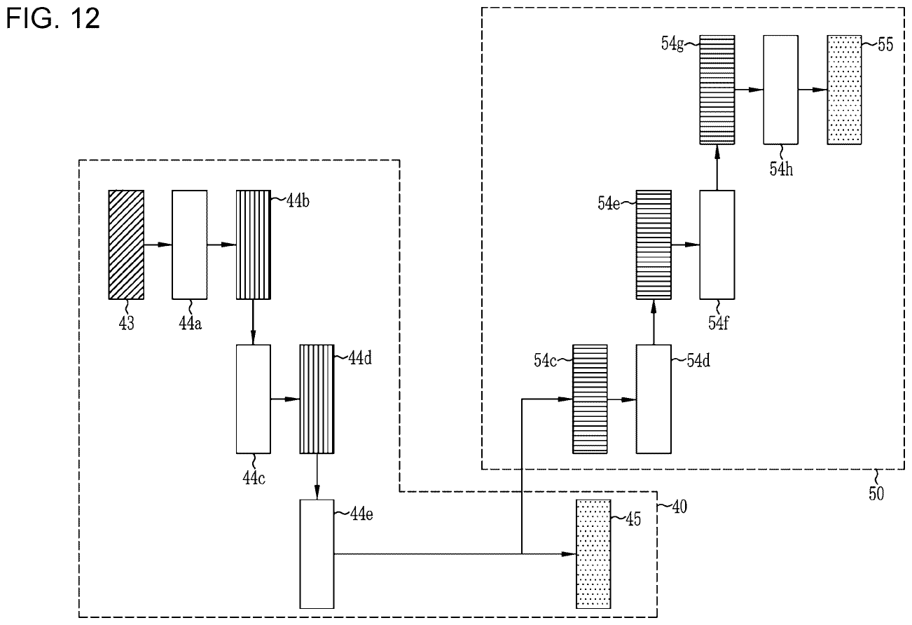

For example, in the learning model 30 as illustrated in FIG. 12, by increasing the number of upsampling layers 54c, 54e, and 54g of the second sub-model 50 to be larger than the number of pooling layers 44b and 44d of the first sub-model 40, the resolution enhancement processing is performed such that the second output image 52 has a higher resolution than the learning input image 21. That is, an example of the learning model 30 of (2) above is illustrated, in which the first sub-model 40 performs the feature extraction and the resolution reduction processing, and the second sub-model 50 performs the resolution enhancement processing such that the second output image 52 has a higher resolution than the learning input image 21. In this case, the resolution of the first intermediate feature map extracted from the convolutional layer 44a of the first sub-model 40 may be enhanced, and the first intermediate feature map may be input to the convolutional layer 54h of the second sub-model 50.

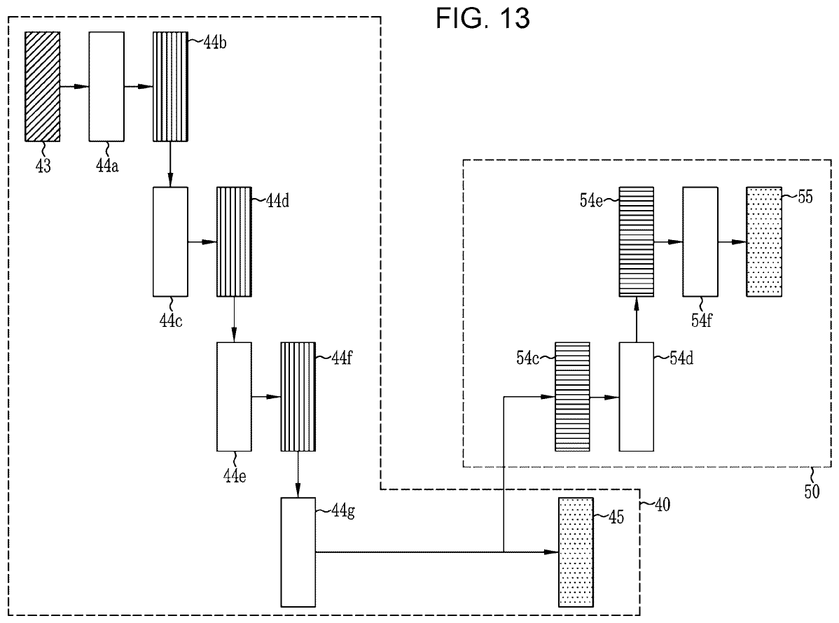

In addition, in the learning model 30 as illustrated in FIG. 13, by decreasing the number of upsampling layers 54c and 54e of the second sub-model 50 to be smaller than the number of pooling layers 44b, 44d, and 44f of the first sub-model 40, the resolution enhancement processing is performed such that the second output image 52 has a lower resolution than the learning input image 21. That is, an example of the learning model 30 of (3) above is illustrated, in which the first sub-model 40 performs the feature extraction and the resolution reduction processing, and the second sub-model 50 performs the resolution enhancement processing such that the second output image 52 has a lower resolution than the learning input image 21 (however, the second output image 52 has a higher resolution than the first output image 42).

Note that although an example in which the learning model 30 has two sub-models is disclosed above, the learning model 30 may have one machine learning model as long as it has a configuration including the input layer 43, the first intermediate layer 44 that extracts the first feature map 41 by feature extraction, the first output layer 45 that outputs the first output image 42 based on the first feature map 41, the second intermediate layer 54 that receives the first feature map 41 and extracts the second feature map 51 by performing resolution enhancement processing on at least the first feature map 41, and the second output layer 55 that outputs the second output image 52 based on the second feature map 51. That is, the learning model 30 disclosed in this embodiment is obtained by configuring the machine learning model in such a manner that an intermediate layer for the feature extraction and an output layer are provided at stages before the intermediate layer for performing the resolution enhancement processing, and another output layer is provided at a stage subsequent to the intermediate layer for performing the resolution enhancement processing.

The learning input image 21 and the inference input image 121 are preferably medical images. The medical image is an image acquired by the modality 15 such as an endoscope, a radiation imaging apparatus, an ultrasound imaging apparatus, or a nuclear magnetic resonance apparatus and used by a doctor or the like for diagnosis. Specifically, there are an endoscopic image, a radiation image such as an X-ray image, a computed tomography (CT) image, an ultrasound image, a magnetic resonance imaging (MRI) image, and the like.

By setting, as the learned model 13, the learning model 30 that performs learning by using a medical image as the learning input image 21 and further making an inference by using the learned model 13 by using a medical image as the inference input image 121, the region of interest in the medical image can be recognized with high accuracy and at high speed, and by supporting diagnosis performed by a user who is a doctor, the accuracy of diagnosis can be improved. In addition, the learning apparatus 11 according to this example can perform learning so as to increase the output accuracy also in the medical field where the amount of image data serving as the learning data set 20 generally tends to be small.

Note that the learning input image 21 and the inference input image 121 may be images other than medical images. For example, the image may be an image acquired using a drive recorder as the modality 15 and including a road, a vehicle, and a person as the subjects.

The inference input image 121 is preferably an image acquired in time-series order. For example, if the modality 15 is a flexible scope to be inserted into a digestive tract of a patient, the inference input image 121 is an endoscopic image that is obtained by capturing an image of a surface of a mucous membrane of the digestive tract and that is acquired in a time-series manner in a process in which a doctor moves a tip part of an endoscope from a rectum to an ileocecal part.

In addition, if the modality 15 is an ultrasound image diagnostic apparatus that emits ultrasound by bringing a probe into contact with the skin of a patient's abdomen, the inference input image 121 is an ultrasound image. The ultrasound image is a medical image acquired while being changed in a time-series manner in accordance with respiration or pulsation of a patient.

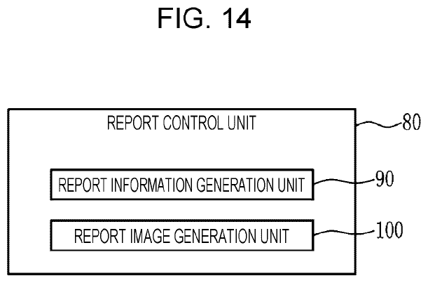

The inference result image 142 output by the learned model 13 of the inference apparatus 12 is transmitted to a report control unit 80 of the image processing apparatus 10 (see FIG. 6). As illustrated in FIG. 14, the report control unit 80 includes a report information generation unit 90 and a report image generation unit 100.

The report information generation unit 90 generates report information based on information obtained by extracting a feature of the inference input image 121, the feature being included in the inference result image 142. The report information is information indicating where a region of interest, which is a feature extracted to the learned model 13, is included in the inference input image 121. The report image generation unit 100 generates a report image, which is an image for displaying the report information, by using the report information.

The report image is preferably a superimposed image in which the report information is superimposed on an image acquired by the modality 15. In addition, there is a sub-image that is an image for displaying the report information at a position different from a position at which the image acquired by the modality 15 is displayed.

The image acquired by the modality 15 is preferably the inference input image 121 or an image acquired later than the inference input image 121 in time series. If the inference result image 142 is output substantially at the same time as the acquisition of the inference input image 121, the position of the region of interest indicated by the report information is substantially the same even in an image acquired later than the inference input image 121 in time series (in particular, immediately after several frames or the like). Thus, even if the report image (superimposed image or sub-image) is generated by using the image acquired later than the inference input image 121 in time series and the report information, a user can recognize the position of the region of interest included in the report information.

The report information is preferably position information of a specific shape surrounding a region indicating a feature included in the inference input image 121 transmitted from the modality 15. The specific shape is, for example, a bounding box surrounding the region of interest. Note that the specific shape is not limited to a rectangle and may be an ellipse or a polygon. In addition, a display mode such as the color of the specific shape may be set as appropriate or may be automatically set. Furthermore, if regions of interest as a plurality of features are detected as a result of segmentation performed by the learned model 13 and the regions of interest are classified into a plurality of classes such as “polyp” and “inflammation”, display modes such as the shape and color of the specific shape may be different for the respective classes. In addition, a class label such as “polyp” or “inflammation” may be displayed near the specific shape.

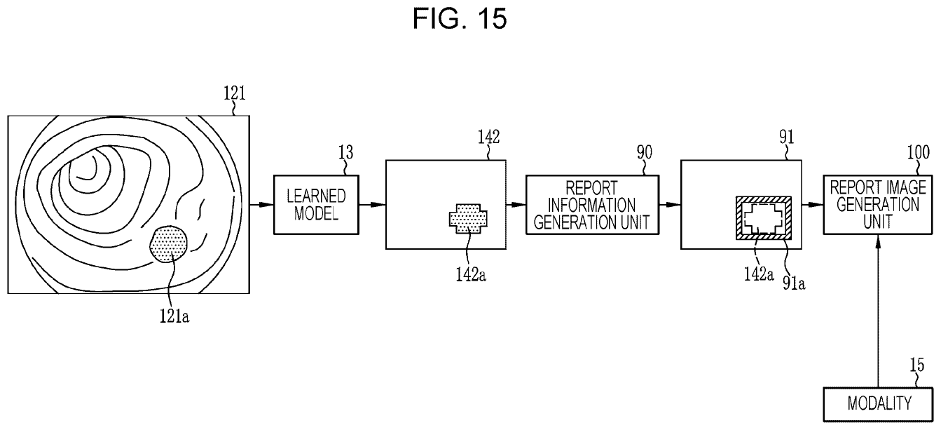

A flow of generation of the report image in a case where the report information is position information of a specific shape surrounding a region indicating a feature included in the inference input image 121 and a specific example of the generated report image will be described. First, a case where the report image is a superimposed image will be exemplified with reference to FIG. 15. In response to the inference input image 121 being input to the learned model 13, the inference result image 142 as the first output image 42 is output. The inference result image 142 includes a region of interest 142a as an extracted feature 121a. In the specific example illustrated in FIG. 15, output of the inference result image 142 having a lower resolution than the inference input image 121 is indicated by a small size of the inference result image 142. In addition, the feature 121a of the inference input image 121 subjected to resolution reduction processing is indicated as being classified as the region of interest 142a.

Subsequently, the report information generation unit 90 generates report information 91 from the inference result image 142. In the specific example illustrated in FIG. 15, the report information 91 is position information of a rectangle 91a surrounding the extracted region of interest 142a. Note that, although the region of interest 142a is indicated by a broken line for description in FIG. 15, the report information generation unit 90 generates only the position information of the rectangle 91a as the report information 91.

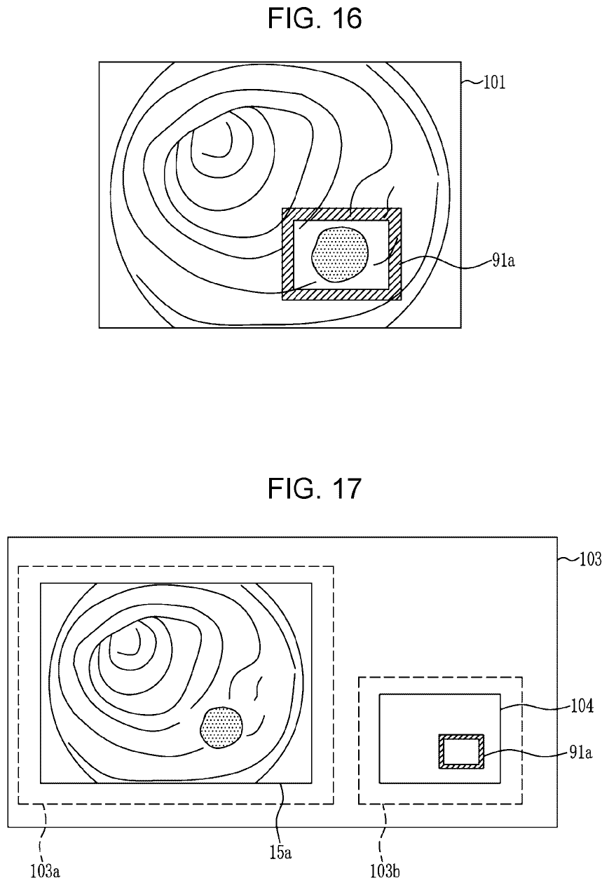

The generated report information 91 is transmitted to the report image generation unit 100. Furthermore, an image from the modality 15 (the inference input image 121 or the image acquired later than the inference input image 121 in time series) is transmitted to the report image generation unit 100. The report image generation unit 100 generates a superimposed image 101 as illustrated in FIG. 16 by superimposing the report information 91 on the image from the modality 15. On the superimposed image 101, the position information of the rectangle 91a is superimposed as the report information 91. The superimposed image 101 is transmitted to a display control unit 110 (see FIG. 6).

The display control unit 110 performs control such that the report image generated by the report image generation unit 100 is displayed on a display 16 (see FIG. 6). Finally, the report image that can be visually recognized by a user is displayed on the display 16.

By displaying the report information 91 as the superimposed image 101 on the display 16 as in the above example, the report information can be recognized without moving the user's line of sight.

Next, a modification will be described in which, as the report image, the report information 91 that is the position information of the rectangle 91a is displayed as a sub-image. The flow until the report information 91 and the image from the modality 15 are transmitted to the report image generation unit 100 is substantially the same as that in the example described with reference to FIG. 15. In this case, as illustrated in FIG. 17, a report image 103 generated by the report image generation unit 100 has a main section 103a for displaying an image 15a from the modality 15 and a sub-section 103b for displaying a sub-image 104 that is an image for displaying the report information 91 (the rectangle 91a indicating the position information of the region of interest 142a). The main section 103a and the sub-section 103b may have any positional relationship as long as they are at different positions on the report image 103. In addition, the sizes of the main section 103a and the sub-section 103b can be set as appropriate. The report image 103 is transmitted to the display control unit 110.

In some situations, it is not preferable to superimpose report information on the image from the modality 15 displayed on the display 16. For example, if a user is a doctor, the user may want to closely observe an image including a region of interest, which is a lesion or the like. In such a situation, if the report information is superimposed on the image, the user's observation is interrupted instead. Thus, by displaying the report information 91 as a sub-image as in the above modification, the position information of the region of interest to be observed can be displayed without interrupting the user's observation.

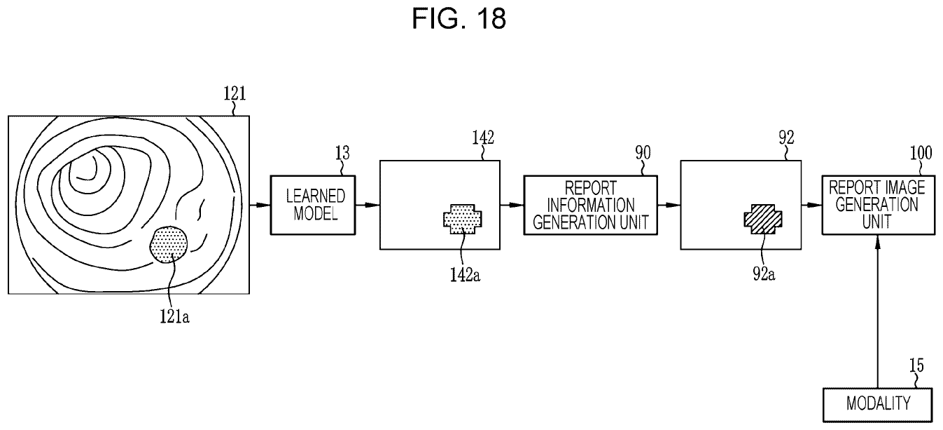

Next, a modification of generating, from the inference input image 121, position information of a small region classified as a region of interest as the report information and generating a report image indicating the position information of the small region in a specific color will be described with reference to a specific example illustrated in FIG. 18. First, an example of generating a superimposed image as the report image will be described. Also in this case, as in the example illustrated in FIG. 15, by inputting the inference input image 121 to the learned model 13, the inference result image 142 including the region of interest 142a as the extracted feature 121a is output and transmitted to the report information generation unit 90.

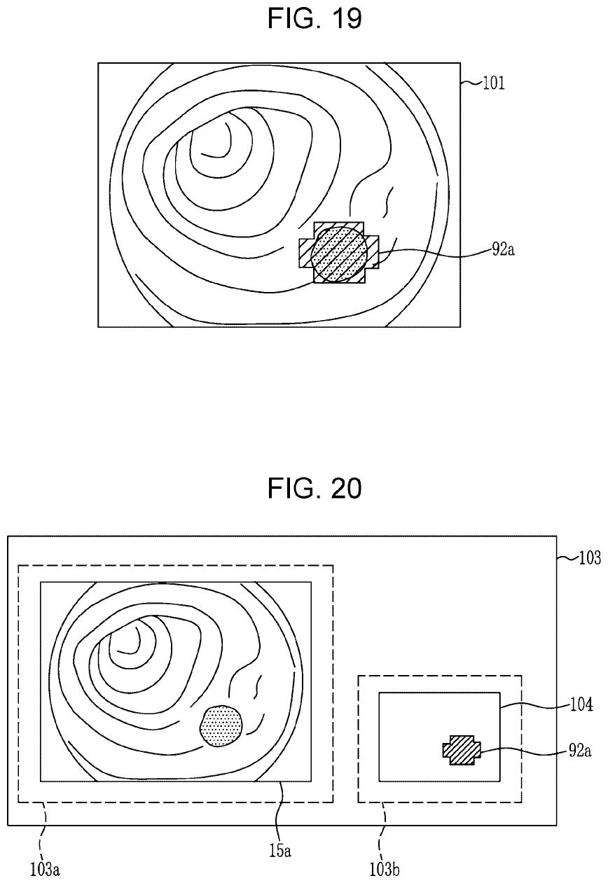

As illustrated in FIG. 18, the report information generation unit 90 generates position information of a small region 92a that is the extracted region of interest 142a as report information 92. As illustrated in FIG. 19, the report image generation unit 100 generates the superimposed image 101 by superimposing, on the image from the modality 15, an image representing the position information of the small region 92a as the report information 92 in a specific color. On the superimposed image 101, the position information of the small region 92a indicated in the specific color is superimposed as the report information 92. The position information of the small region 92a indicated in the specific color is preferably superimposed by adjusting the transparency such that the image from the modality 15, which is the background, is seen through. The superimposed image 101 is transmitted to the display control unit 110. Note that any color can be set as the specific color in accordance with the modality 15. With the above configuration, it is possible to cause a user to recognize the region of interest as a color distribution.

Furthermore, a modification will be described in which, as the report image, the report information 92 that is the position information of the small region 92a indicated in a specific color is displayed as a sub-image. The flow until the report information 92 and the image from the modality 15 are transmitted to the report image generation unit 100 is the same as that in the example described with reference to FIG. 18. In this case, as illustrated in FIG. 20, in the report image 103, the image 15a from the modality 15 is displayed in the main section 103a, and the report information 92 is displayed as the sub-image 104 in the sub-section 103b. The sub-image 104 is preferably a mini-map indicating the position information of the small region 92a in a specific color. With the above configuration, it is possible to visualize the distribution of the region of interest and cause a user to recognize the distribution of the region of interest without interrupting the user's observation.

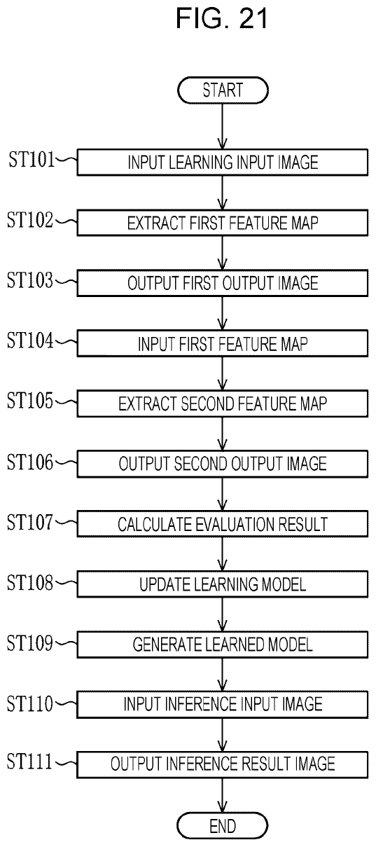

A sequential flow of an operation method in the image processing apparatus 10 according to this embodiment will be described with reference to the flowchart in FIG. 21. First, the learning input image 21 is input to the first sub-model 40 of the learning model 30 (step ST101). The first feature map 41 is extracted from the learning input image 21 by using the first sub-model 40 (step ST102), and the first output image 42 is output based on the first feature map 41 (step ST103). Subsequently, the first feature map 41 is input to the second sub-model 50 (step ST104). The second feature map 51 is extracted from the first feature map 41 by using the second sub-model 50 (step ST105), and the second output image 52 having higher resolution than the first output image 42 is output based on the second feature map 51 (step ST106).

Subsequently, the evaluation unit 60 calculates the evaluation result 61 by using the second output image 52 (step ST107). The update unit 70 updates the parameters of the learning model 30 by using the evaluation result 61 (step ST108). Through repeated updating, the learning model 30 is generated as the learned model 13 (step ST109). Finally, by inputting the inference input image 121 to the learned model 13 that has completed learning (step ST110), the inference processing of the learned model 13 is performed, and the first output image 42 as the inference result image 142 is output from the learned model 13 (step ST111).

In the present embodiment, an “image” refers to image data. The image data includes the learning input image 21, the learning correct answer image 22, the inference input image 121, the inference result image 142, the first output image 42, the second output image 52, the first feature map 41, the second feature map 51, the first intermediate feature map, the second intermediate feature map, the correct answer label image, the first correct answer label image 24, the second correct answer label image 25, the image from the modality 15, the report images 101 and 103, and the sub-image 104.

In the image processing apparatus 10, programs relating to various processes, controls, or the like are incorporated in a program storage memory (not illustrated). A control unit (not illustrated) configured by a processor operates a program incorporated in the program storage memory to implement the functions of the learning apparatus 11, the inference apparatus 12, the report control unit 80, and the display control unit 110. Note that the learning apparatus 11 may be separated from the image processing apparatus 10, and in this case, the learning apparatus 11 may include a first control unit configured by a processor, and the image processing apparatus 10 may include a second control unit configured by a processor.

In the above embodiment, a hardware configuration of a processing unit that performs various processes, such as the learning apparatus 11, the inference apparatus 12, the report control unit 80, the display control unit 110, or the control unit, is any of the following various processors. Various processors include a central processing unit (CPU) that is a general-purpose processor functioning as various processing units by executing software (programs), a programmable logic device (PLD) that is a processor in which the circuit configuration is changeable after manufacture, such as field programmable gate array (FPGA), a dedicated electric circuit that is a processor having a circuit configuration that is specially designed to execute various processes, and the like.

One processing unit may be constituted by one of these various processors, or may be constituted by two or more processors of the same type or different types in combination (e.g., a combination of a plurality of FPGAs or a combination of a CPU and an FPGA). In addition, a plurality of processing units may be constituted by one processor. Firstly, as an example of constituting a plurality of processing units by one processor, there is a form in which one processor is constituted by a combination of one or more CPUs and software, and the processor functions as a plurality of processing units, as typified by a computer such as a client or a server. Secondly, there is a form using a processor that implements the functions of the entire system including a plurality of processing units by using one integrated circuit (IC) chip, as typified by a system on chip (SoC) or the like. In this manner, various processing units are constituted by one or more of the above various processors in terms of hardware configuration.

More specifically, the hardware configuration of these various processors is electric circuitry constituted by a combination of circuit elements such as semiconductor elements. The hardware configuration of the storage unit is a storage device such as a hard disc drive (HDD) or a solid state drive (SSD).

### REFERENCE SIGNS LIST

10 image processing apparatus
11 learning apparatus
12 inference apparatus
13 learned model
14 data storage unit
15 modality
15a image from modality
16 display
20 learning data set
21 learning input image
22 learning correct answer image
22a, 22b, 22c, 22d, 22e, 92a small region
23a, 23b, 23c, 23d, 23e correct answer label
24 first correct answer label image
25 second correct answer label image
30 learning model
40 first sub-model
41 first feature map
41a, 42a, 52a, 142a region of interest
41b first intermediate feature map
42 first output image
42b, 52b region other than region of interest
43 input layer
44 first intermediate layer
44a, 44c, 44e, 44g, 54b, 54d, 54f, 54h convolutional layer
44b, 44d, 44f pooling layer
45 first output layer
50 second sub-model
51 second feature map
52 second output image
55 second intermediate layer
54a, 54c, 54e, 54g upsampling layer
55 second output layer
60 evaluation unit
61 evaluation result
62 first evaluation result
63 second evaluation result
70 update unit
80 report control unit
90 report information generation unit
91, 92 report information
91a rectangle
100 report image generation unit
101 superimposed image
103 report image
103a main section
103b sub-section
104 sub-image
110 display control unit
121 inference input image
121a feature
142 inference result image

What is claimed is:

1. An image processing apparatus comprising:
a processor configured to:
output a first output image based on a first feature map extracted by inputting a learning input image to a first sub-model in a learning model including the first sub-model and a second sub-model;
output a second output image having a higher resolution than the first output image, based on a second feature map extracted by inputting the first feature map to the second sub-model;
calculate an evaluation result by using the second output image;
update the learning model by using the evaluation result to set the learning model as a learned model including a first sub-learned model that is the first sub-model that has performed learning and a second sub-learned model that is the second sub-model that has performed learning; and
output the first output image as an inference result image based on the first feature map extracted by inputting an inference input image to the first sub-learned model in the learned model.

2. The image processing apparatus according to claim 1, wherein
the processor is configured to calculate the evaluation result by comparing the second output image with a learning correct answer image corresponding to the learning input image, and
the learning correct answer image is a correct answer label image in which a correct answer label is attached for each of regions constituting the learning correct answer image.

3. The image processing apparatus according to claim 2, wherein the processor is configured to:
calculate a first evaluation result as the evaluation result by comparing the first output image with a first correct answer label image as the correct answer label image having a resolution of the first output image, and calculate a second evaluation result as the evaluation result by comparing the second output image with a second correct answer label image as the correct answer label image having the resolution of the second output image; and
update the learning model by using the first evaluation result and the second evaluation result.

4. The image processing apparatus according to claim 3, wherein the first correct answer label image is generated by performing resolution reduction processing on the second correct answer label image.

5. The image processing apparatus according to claim 1, wherein the resolution of the second output image is same as a resolution of the learning input image.

6. The image processing apparatus according to claim 1, wherein the resolution of the second output image is lower than a resolution of the learning input image.

7. The image processing apparatus according to claim 1, wherein the first sub-model and the second sub-model are constituted by using a convolutional neural network.

8. The image processing apparatus according to claim 1, wherein a resolution of the first output image is lower than a resolution of the learning input image.

9. The image processing apparatus according to claim 1, wherein the processor is configured to:
further output an intermediate feature map having a higher resolution than the first feature map by using the first sub-model; and
further input the intermediate feature map to the second sub-model.

10. The image processing apparatus according to claim 1, wherein the learning input image and the inference input image are medical images.

11. The image processing apparatus according to claim 1, wherein the inference input image is an image acquired in time-series order.

12. The image processing apparatus according to claim 1, wherein the processor is configured to:
generate report information based on information of the inference result image;
generate a report image based on the report information; and
perform control to display the report image.

13. The image processing apparatus according to claim 12, wherein the report image is generated to display the report information so as to be superimposed on the inference input image or an image acquired later than the inference input image in time series.

14. The image processing apparatus according to claim 12, wherein the report image is generated so as to display the inference input image or an image acquired later than the inference input image in time series and the report information at positions different from each other.

15. The image processing apparatus according to claim 13, wherein the report information is position information of a specific shape surrounding a region indicating a feature included in the inference input image.

16. An operation method for an image processing apparatus, the operation method comprising steps of:
outputting a first output image based on a first feature map extracted by inputting a learning input image to a first sub-model in a learning model including the first sub-model and a second sub-model;
outputting a second output image having a higher resolution than the first output image, based on a second feature map extracted by inputting the first feature map to the second sub-model;
calculating an evaluation result by using the second output image;
updating the learning model by using the evaluation result to set the learning model as a learned model including a first sub-learned model that is the first sub-model that has performed learning and a second sub-learned model that is the second sub-model that has performed learning; and
outputting the first output image as an inference result image based on the first feature map extracted by inputting an inference input image to the first sub-learned model in the learned model.

17. An inference apparatus comprising:
a processor configured to output a first output image as an inference result image, based on a first feature map extracted by inputting an inference input image to a first sub-learned model in a learned model including the first sub-learned model and a second sub-learned model, wherein
the learned model is generated by setting, in a learning model including a first sub-model and a second sub-model, the first sub-model as the first sub-learned model and the second sub-model as the second sub-learned model, and
the learning model outputs a first output image based on the first feature map extracted based on a learning input image input to the first sub-model, outputs a second output image having a higher resolution than the first output image, based on a second feature map extracted based on the first feature map input to the second sub-model, and is updated by using an evaluation result calculated using the second output image to perform learning.

18. A learning apparatus comprising:
a processor configured to:
output a first output image based on a first feature map extracted by inputting a learning input image to a first sub-model in a learning model including the first sub-model and a second sub-model;
output a second output image having a higher resolution than the first output image, based on a second feature map extracted by inputting the first feature map to the second sub-model;
calculate an evaluation result by using the second output image; and
update the learning model by using the evaluation result to perform learning, wherein
the resolution of the second output image is lower than a resolution of the learning input image.
